(19) United States
(12) Patent Application Publication
(10) Pub. No.: US 2022/0238203 A1
(43) Pub. Date: Jul. 28, 2022

(54) ADAPTIVE NAVIGATION AND REGISTRATION INTERFACE FOR MEDICAL IMAGING

(71) Applicant: Biosense Webster (Israel) Ltd., Yohneam (IL)

(72) Inventors: Helen Wolfson, Haifa (IL); Iris Segal, Qiryat Motzkin (IL); Yoav Pinsky, Bet Keshet (IL); George Gusein, Akko (IL); Uriel Hod, Ein Ayala (IL)

(73) Assignee: Biosense Webster (Israel) Ltd., Yokneam (IL)

(21) Appl. No.: 17/158,510

(22) Filed: Jan. 26, 2021

(51) Int. Cl. G16H 20/40; G06N 20/00; G16H 10/60; G16H 30/20

(52) U.S. Cl. CPC G16H 20/40; G16H 30/20; G16H 10/60; G06N 20/00

(57) ABSTRACT

A method is provided. The method is implemented by an interface engine stored as processor executable code on a memory coupled to a processor. The method includes aggregating data from completed cases, analyzing the data for accuracy, consistency, or error within or across the one completed cases, and generating one or more grades based on the analysis of the data. Note that the data includes location information and registration information, and the completed cases include at least one ear, nose, and throat navigation and registration procedure.

### FIELD OF INVENTION

The present invention is related to a machine learning and/or an artificial intelligence method and system for signal processing and medical imaging. More particularly, the present invention relates to a machine learning/artificial intelligence algorithm that provides an adaptive navigation and registration interface for medical imaging.

### BACKGROUND

Currently, ear, nose, and throat (ENT) navigation systems provide real-time visual confirmation from beginning to end for ENT procedures. For instance, ENT navigation systems provide planning points that help identify drainage pathways, challenging anatomy, and structural anomalies and that can function as beacons to alert ENT physicians when a navigated surgical device approaches the point. Other features of ENT navigation systems include providing an unlimited number of virtual cameras in areas of interest (e.g., allowing the ENT physicians to see beyond an endoscope), a real-time imaging tool that documents surgical changes to the anatomy, an automatic merging feature between computerized tomography (CT) scans and magnetic resonance (MR) scans (e.g., enables blending level control between both scans, while simultaneously navigating), and server connectivity to load scans directly from a network. These features and more are provided by a graphic user interface (“GUI”) of the ENT navigation systems to the ENT physicians. However, present GUIs are limited, and it may be beneficial for ENT physicians to provide an improved graphic user interface (“GUI”) for implementation with any anatomical navigation system to provide enhanced ability for analyzing data and reviewing visualization and guidance for ENT procedures.

### SUMMARY

According to an embodiment, a method is provided. The method is implemented by an interface engine stored as processor executable code on a memory coupled to a processor. The method includes aggregating data from completed cases, analyzing the data for accuracy, consistency, or error within or across the one completed cases, and generating one or more grades based on the analysis of the data. Note that the data can include location information and registration information, and the completed cases can include at least one ear, nose, and throat navigation and registration procedure.

According to one or more embodiments, the method embodiment above can be implemented as an apparatus, a system, and/or a computer program product.

### BRIEF DESCRIPTION OF THE DRAWINGS

A more detailed understanding may be had from the following description, given by way of example in conjunction with the accompanying drawings, wherein like reference numerals in the figures indicate like elements, and wherein:

FIG. 1 illustrates a diagram of an exemplary system in which one or more features of the disclosure subject matter can be implemented according to one or more embodiments;

FIG. 2 illustrates a block diagram of an example system for adaptive navigation and registration interface for medical imaging according to one or more embodiments;

FIG. 3 illustrates an exemplary method according to one or more embodiments;

FIG. 4 illustrates a graphical depiction of an artificial intelligence system according to one or more embodiments;

FIG. 5 illustrates an example of a neural network and a block diagram of a method performed in the neural network according to one or more embodiments;

FIG. 6 illustrates an exemplary method according to one or more embodiments;

FIG. 7 illustrates an exemplary interface according to one or more embodiments;

FIG. 8 illustrates an exemplary interface according to one or more embodiments;

FIG. 9 illustrates an exemplary interface according to one or more embodiments;

FIG. 10 illustrates an exemplary interface according to one or more embodiments; and

FIG. 11 illustrates an exemplary interface according to one or more embodiments.

### DETAILED DESCRIPTION

Disclosed herein is a machine learning and/or an artificial intelligence method and system for signal processing and medical imaging. More particularly, the present invention relates to a machine learning/artificial intelligence algorithm that provides an adaptive navigation and registration interface for medical imaging. For example, the machine learning/artificial intelligence algorithm is a processor executable code or software that is necessarily rooted in process operations by, and in processing hardware of, medical device equipment.

According to an exemplary embodiment, the machine learning/artificial intelligence algorithm can be embodied in an interface engine, which generally aggregates data from completed cases, analyzes the data, and outputs grades for the data. Completed cases can include, but are not limited to, medical treatments, surgical plans, surgical procedures, or medicals diagnoses performed by operations of the interface engine, with ENT navigation and registrations procedures being used as an example herein. Navigation can include a process of determining a location (e.g., an x-y-z coordinate) with respect to an anatomical structure. Registration can include a process of acquiring and maintaining information at each location. The grade indicates ‘how well’ navigation and registration went for each completed case (e.g., that is gathered and analyzed). Further, the graded data can be stored, curated, and analyzed by the interface engine for best practices (e.g., determine what has worked in the past before beginning a new case). Accordingly, one or more advantages, technical effects, and benefits of the interface engine include providing physicians and medical personnel recommendations. For instance, if there is a condition and a plan for treating the condition, the interface engine can compare treatments to provide recommendations (e.g., if a first plan/treatment has a same or higher rate of success than another plan/treatment, then the interface engine can suggest the first plan/treatment).

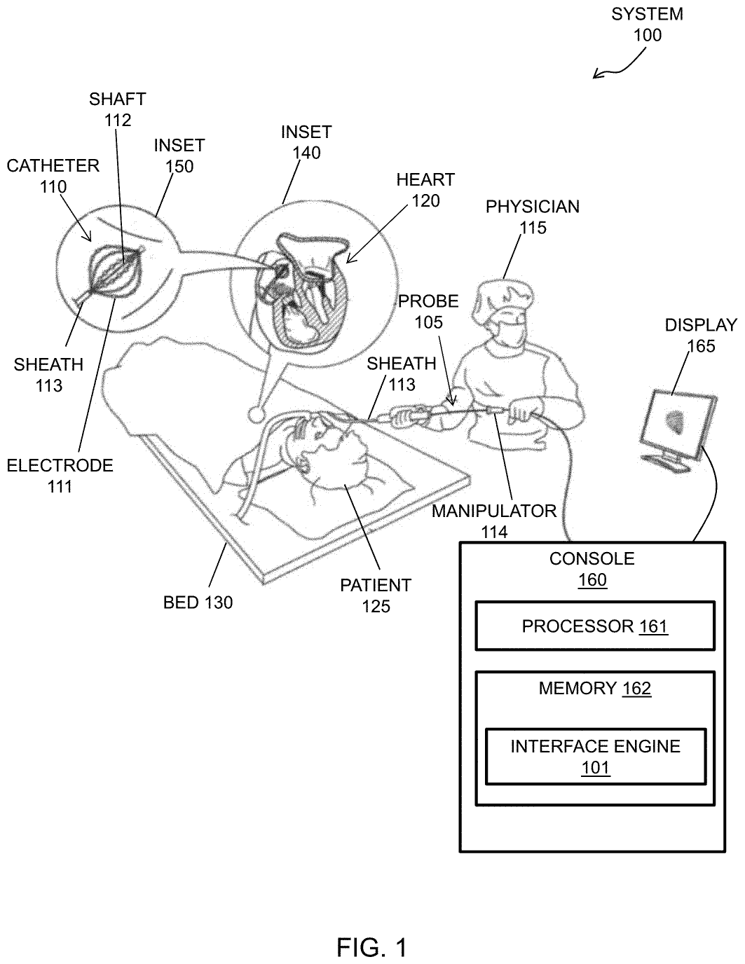

FIG. 1 is a diagram of a system 100 (e.g., medical device equipment, such as ENT navigation systems or other surgical systems) in which one or more features of the subject matter herein can be implemented according to one or more embodiments. All or part of the system 100 can be used to collect information (e.g., biometric data and/or a training dataset) and/or used to implement a machine learning and/or an artificial intelligence algorithm (e.g., an interface engine 101) as described herein.

The system 100, as illustrated, includes a probe 105 with a catheter 110 (including at least one electrode 111), a shaft 112, a sheath 113, and a manipulator 114. The system 100, as illustrated, also includes a physician 115 (or a medical professional or clinician), a heart 120, a patient 125, and a bed 130 (or a table). For ease of explanation, the interface engine 101 of FIG. 1 is described herein with respect to mapping the heart 120; however, any anatomical structure, body part, organ, or portion thereof can be a target for mapping by the interface engine described herein. Note that insets 140 and 150 show the heart 120 and the catheter 110 in greater detail. The system 100 also, as illustrated, includes a console 160 (including one or more processors 161 and memories 162) and a display 165. Note further each element and/or item of the system 100 is representative of one or more of that element and/or that item. The example of the system 100 shown in FIG. 1 can be modified to implement the embodiments disclosed herein. The disclosed embodiments can similarly be applied using other system components and settings. Additionally, the system 100 can include additional components, such as elements for sensing electrical activity, wired or wireless connectors, processing and display devices, or the like.

The system 100 can be utilized to detect, diagnose, and/or treat cardiac conditions (e.g., using the interface engine 101). Cardiac conditions, such as cardiac arrhythmias, persist as common and dangerous medical ailments, especially in the aging population. For instance, the system 100 can be part of a surgical system (e.g., CARTO® system sold by Biosense Webster) that is configured to obtain biometric data (e.g., anatomical and electrical measurements of a patient's organ, such as the heart 120) and perform a cardiac ablation procedure. More particularly, treatments for cardiac conditions such as cardiac arrhythmia often require obtaining a detailed mapping of cardiac tissue, chambers, veins, arteries and/or electrical pathways. For example, a prerequisite for performing a catheter ablation (as described herein) successfully is that the cause of the cardiac arrhythmia is accurately located in a chamber of the heart 120. Such locating may be done via an electrophysiological investigation during which electrical potentials are detected spatially resolved with a mapping catheter (e.g., the catheter 110) introduced into the chamber of the heart 120. This electrophysiological investigation, the so-called electro-anatomical mapping, thus provides 3D mapping data which can be displayed on a monitor. In many cases, the mapping function and a treatment function (e.g., ablation) are provided by a single catheter or group of catheters such that the mapping catheter also operates as a treatment (e.g., ablation) catheter at the same time. In this case, the interface engine 101 can be directly stored and executed by the catheter 110.

In patients (e.g., the patient 125) with normal sinus rhythm (NSR), the heart (e.g., the heart 120), which includes atrial, ventricular, and excitatory conduction tissue, is electrically excited to beat in a synchronous, patterned fashion. Note that this electrical excitement can be detected as intracardiac electrocardiogram (IC ECG) data or the like.

In patients (e.g., the patient 125) with a cardiac arrhythmia (e.g., atrial fibrillation or aFib), abnormal regions of cardiac tissue do not follow a synchronous beating cycle associated with normally conductive tissue, which is in contrast to patients with NSR. Instead, the abnormal regions of cardiac tissue aberrantly conduct to adjacent tissue, thereby disrupting the cardiac cycle into an asynchronous cardiac rhythm. Note that this asynchronous cardiac rhythm can also be detected as the IC ECG data. Such abnormal conduction has been previously known to occur at various regions of the heart 120, for example, in the region of the sino-atrial (SA) node, along the conduction pathways of the atrioventricular (AV) node, or in the cardiac muscle tissue forming the walls of the ventricular and atrial cardiac chambers.

In support of the system 100 detecting, diagnosing, and/or treating cardiac conditions, the probe 105 can be navigated by the physician 115 into the heart 120 of the patient 125 lying on the bed 130. For instance, the physician 115 can insert the shaft 112 through the sheath 113, while manipulating a distal end of the shaft 112 using the manipulator 114 near the proximal end of the catheter 110 and/or deflection from the sheath 113. As shown in an inset 140, the catheter 110 can be fitted at the distal end of the shaft 112. The catheter 110 can be inserted through the sheath 113 in a collapsed state and can be then expanded within the heart 120.

Generally, electrical activity at a point in the heart 120 may be typically measured by advancing the catheter 110 containing an electrical sensor at or near its distal tip (e.g., the at least one electrode 111) to that point in the heart 120, contacting the tissue with the sensor and acquiring data at that point. One drawback with mapping a cardiac chamber using a catheter type containing only a single, distal tip electrode is the long period of time required to accumulate data on a point-by-point basis over the requisite number of points required for a detailed map of the chamber as a whole. Accordingly, multiple-electrode catheters (e.g., the catheter 110) have been developed to simultaneously measure electrical activity at multiple points in the heart chamber.

The catheter 110, which can include the at least one electrode 111 and a catheter needle coupled onto a body thereof, can be configured to obtain biometric data, such as electrical signals of an intra-body organ (e.g., the heart 120), and/or to ablate tissue areas of thereof (e.g., a cardiac chamber of the heart 120). Note that the electrodes 111 are representative of any like elements, such as tracking coils, piezoelectric transducer, electrodes, or combination of elements configured to ablate the tissue areas or to obtain the biometric data. According to one or more embodiments, the catheter 110 can include one or more position sensors that used are to determine trajectory information. The trajectory information can be used to infer motion characteristics, such as the contractility of the tissue.

Biometric data (e.g., patient biometrics, patient data, or patient biometric data) can include one or more of local time activations (LATs), electrical activity, topology, bipolar mapping, reference activity, ventricle activity, dominant frequency, impedance, or the like. The LAT can be a point in time of a threshold activity corresponding to a local activation, calculated based on a normalized initial starting point. Electrical activity can be any applicable electrical signals that can be measured based on one or more thresholds and can be sensed and/or augmented based on signal to noise ratios and/or other filters. A topology can correspond to the physical structure of a body part or a portion of a body part and can correspond to changes in the physical structure relative to different parts of the body part or relative to different body parts. A dominant frequency can be a frequency or a range of frequency that is prevalent at a portion of a body part and can be different in different portions of the same body part. For example, the dominant frequency of a PV of a heart can be different than the dominant frequency of the right atrium of the same heart. Impedance can be the resistance measurement at a given area of a body part.

Examples of biometric data include, but are not limited to, patient identification data, IC ECG data, bipolar intracardiac reference signals, anatomical and electrical measurements, trajectory information, body surface (BS) ECG data, historical data, brain biometrics, blood pressure data, ultrasound signals, radio signals, audio signals, a two- or three-dimensional image data, blood glucose data, and temperature data. The biometrics data can be used, generally, to monitor, diagnosis, and treatment any number of various diseases, such as cardiovascular diseases (e.g., arrhythmias, cardiomyopathy, and coronary artery disease) and autoimmune diseases (e.g., type I and type II diabetes). Note that BS ECG data can include data and signals collected from electrodes on a surface of a patient, IC ECG data can include data and signals collected from electrodes within the patient, and ablation data can include data and signals collected from tissue that has been ablated. Further, BS ECG data, IC ECG data, and ablation data, along with catheter electrode position data, can be derived from one or more procedure recordings.

For example, the catheter 110 can use the electrodes 111 to implement intravascular ultrasound and/or MRI catheterization to image the heart 120 (e.g., obtain and process the biometric data). Inset 150 shows the catheter 110 in an enlarged view, inside a cardiac chamber of the heart 120. Although the catheter 110 is shown to be a point catheter, it will be understood that any shape that includes one or more electrodes 111 can be used to implement the embodiments disclosed herein.

Examples of the catheter 106 include, but are not limited to, a linear catheter with multiple electrodes, a balloon catheter including electrodes dispersed on multiple spines that shape the balloon, a lasso or loop catheter with multiple electrodes, or any other applicable shape. Linear catheters can be fully or partially elastic such that it can twist, bend, and or otherwise change its shape based on received signal and/or based on application of an external force (e.g., cardiac tissue) on the linear catheter. The balloon catheter can be designed such that when deployed into a patient's body, its electrodes can be held in intimate contact against an endocardial surface. As an example, a balloon catheter can be inserted into a lumen, such as a pulmonary vein (PV). The balloon catheter can be inserted into the PV in a deflated state, such that the balloon catheter does not occupy its maximum volume while being inserted into the PV. The balloon catheter can expand while inside the PV, such that those electrodes on the balloon catheter are in contact with an entire circular section of the PV. Such contact with an entire circular section of the PV, or any other lumen, can enable efficient imaging and/or ablation.

According to other examples, body patches and/or body surface electrodes may also be positioned on or proximate to a body of the patient 125. The catheter 110 with the one or more electrodes 111 can be positioned within the body (e.g., within the heart 120) and a position of the catheter 110 can be determined by the 100 system based on signals transmitted and received between the one or more electrodes 111 of the catheter 110 and the body patches and/or body surface electrodes. Additionally, the electrodes 111 can sense the biometric data (e.g., LAT values) from within the body of the patient 125 (e.g., within the heart 120). The biometric data can be associated with the determined position of the catheter 110 such that a rendering of the patient's body part (e.g., the heart 120) can be displayed and show the biometric data overlaid on a shape of the body part.

The probe 105 and other items of the system 100 can be connected to the console 160. The console 160 can include any computing device, which employs the machine learning and/or an artificial intelligence algorithm (represented as the interface engine 101). According to an embodiment, the console 160 includes the one or more processors 161 (any computing hardware) and the memory 162 (any non-transitory tangible media), where the one or more processors 161 execute computer instructions with respect the interface engine 101 and the memory 162 stores these instructions for execution by the one or more processors 161. For instance, the console 160 can be configured to receive and process the biometric data and determine if a given tissue area conducts electricity. In some embodiments, the console 160 can be further programmed by the interface engine 101 (in software) to carry out the functions of aggregating data from completed cases, analyzing the data for accuracy, consistency, or error within or across the one completed cases, and generating one or more grades based on the analysis of the data. According to one or more embodiments, the interface engine 101 can be external to the console 160 and can be located, for example, in the catheter 110, in an external device, in a mobile device, in a cloud-based device, or can be a standalone processor. In this regard, the interface engine 101 can be transferable/downloaded in electronic form, over a network.

In an example, the console 160 can be any computing device, as noted herein, including software (e.g., the interface engine 101) and/or hardware (e.g., the processor 161 and the memory 162), such as a general-purpose computer, with suitable front end and interface circuits for transmitting and receiving signals to and from the probe 105, as well as for controlling the other components of the system 100. For example, the front end and interface circuits include input/output (I/O) communication interfaces that enables the console 160 to receive signals from and/or transfer signals to the at least one electrode 111. The console 160 can include real-time noise reduction circuitry typically configured as a field programmable gate array (FPGA), followed by an analog-to-digital (A/D) ECG or electrocardiograph/electromyogram (EMG) signal conversion integrated circuit. The console 160 can pass the signal from an A/D ECG or EMG circuit to another processor and/or can be programmed to perform one or more functions disclosed herein.

The display 165, which can be any electronic device for the visual presentation of the biometric data, is connected to the console 160. According to an embodiment, during a procedure, the console 160 can facilitate on the display 165 a presentation of a body part rendering to the physician 115 and store data representing the body part rendering in the memory 162. For instance, maps depicting motion characteristics can be rendered/constructed based on the trajectory information sampled at a sufficient number of points in the heart 120. Further, the display 165 in conjunction with the interface engine 101 can provide errors during a case via graphical representations, where X axis is a timeline, provide separate plots that represent different systems/ports/tools, provide graphical presentations of registration (e.g., including color coding of registration quality), and provide graphical presentations of navigation (e.g., color coded by time and size coded by type of tool). The interface engine 101 can further render a movie, which wraps up an entire case in seconds (e.g., seven seconds) and shows a 360 degree panorama of a navigation map, which allows replay.

As an example, the display 165 can include a touchscreen that can be configured to accept inputs from the medical professional 115, in addition to presenting the body part rendering.

In some embodiments, the physician 115 can manipulate the elements of the system 100 and/or the body part rendering using one or more input devices, such as a touch pad, a mouse, a keyboard, a gesture recognition apparatus, or the like. For example, an input device can be used to change a position of the catheter 110, such that rendering is updated. Note that the display 165 can be located at a same location or a remote location, such as a separate hospital or in separate healthcare provider networks.

According to one or more embodiments, the system 100 can also obtain the biometric data using ultrasound, computed tomography (CT), MRI, or other medical imaging techniques utilizing the catheter 110 or other medical equipment. For instance, the system 100 can obtain ECG data and/or anatomical and electrical measurements of the heart 120 (e.g., the biometric data) using one or more catheters 110 or other sensors. More particularly, the console 160 can be connected, by a cable, to BS electrodes, which include adhesive skin patches affixed to the patient 125. The BS electrodes can procure/generate the biometric data in the form of the BS ECG data. For instance, the processor 161 can determine position coordinates of the catheter 110 inside the body part (e.g., the heart 120) of the patient 125. The position coordinates may be based on impedances or electromagnetic fields measured between the body surface electrodes and the electrode 111 of the catheter 110 or other electromagnetic components. Additionally, or alternatively, location pads may be located on a surface of the bed 130 and may be separate from the bed 130. The biometric data can be transmitted to the console 160 and stored in the memory 162. Alternatively, or in addition, the biometric data may be transmitted to a server, which may be local or remote, using a network as further described herein.

According to one or more embodiments, the catheter 110 may be configured to ablate tissue areas of a cardiac chamber of the heart 120. Inset 150 shows the catheter 110 in an enlarged view, inside a cardiac chamber of the heart 120. For instance, ablation electrodes, such as the at least one electrode 111, may be configured to provide energy to tissue areas of an intra-body organ (e.g., the heart 120). The energy may be thermal energy and may cause damage to the tissue area starting from the surface of the tissue area and extending into the thickness of the tissue area. The biometric data with respect to ablation procedures (e.g., ablation tissues, ablation locations, etc.) can be considered ablation data.

According to an example, with respect to obtaining the biometric data, a multi-electrode catheter (e.g., the catheter 110) can be advanced into a chamber of the heart 120. Anteroposterior (AP) and lateral fluorograms can be obtained to establish the position and orientation of each of the electrodes. ECGs can be recorded from each of the electrodes 111 in contact with a cardiac surface relative to a temporal reference, such as the onset of the P-wave in sinus rhythm from a BS ECG. The system, as further disclosed herein, may differentiate between those electrodes that register electrical activity and those that do not due to absence of close proximity to the endocardial wall. After initial ECGs are recorded, the catheter may be repositioned, and fluorograms and ECGs may be recorded again. An electrical map (e.g., via cardiac mapping) can then be constructed from iterations of the process above.

Cardiac mapping can be implemented using one or more techniques. Generally, mapping of cardiac areas such as cardiac regions, tissue, veins, arteries and/or electrical pathways of the heart 120 may result in identifying problem areas such as scar tissue, arrhythmia sources (e.g., electric rotors), healthy areas, and the like. Cardiac areas may be mapped such that a visual rendering of the mapped cardiac areas is provided using a display, as further disclosed herein. Additionally, cardiac mapping (which is an example of heart imaging) may include mapping based on one or more modalities such as, but not limited to local activation time (LAT), an electrical activity, a topology, a bipolar mapping, a dominant frequency, or an impedance. Data (e.g., biometric data) corresponding to multiple modalities may be captured using a catheter (e.g., the catheter 110) inserted into a patient's body and may be provided for rendering at the same time or at different times based on corresponding settings and/or preferences of the physician 115.

As an example of a first technique, cardiac mapping may be implemented by sensing an electrical property of heart tissue, for example, LAT, as a function of the precise location within the heart 120. The corresponding data (e.g., biometric data) may be acquired with one or more catheters (e.g., the catheter 110) that are advanced into the heart 1120 and that have electrical and location sensors (e.g., the electrodes 111) in their distal tips. As specific examples, location and electrical activity may be initially measured on about 10 to about 20 points on the interior surface of the heart 120. These data points may be generally sufficient to generate a preliminary reconstruction or map of the cardiac surface to a satisfactory quality. The preliminary map may be combined with data taken at additional points to generate a more comprehensive map of the heart's electrical activity. In clinical settings, it is not uncommon to accumulate data at 100 or more sites to generate a detailed, comprehensive map of heart chamber electrical activity. The generated detailed map may then serve as the basis for deciding on a therapeutic course of action, for example, tissue ablation as described herein, to alter the propagation of the heart's electrical activity and to restore normal heart rhythm.

Further, cardiac mapping can be generated based on detection of intracardiac electrical potential fields (e.g., which is an example of IC ECG data and/or bipolar intracardiac reference signals). A non-contact technique to simultaneously acquire a large amount of cardiac electrical information may be implemented. For example, a catheter type having a distal end portion may be provided with a series of sensor electrodes distributed over its surface and connected to insulated electrical conductors for connection to signal sensing and processing means. The size and shape of the end portion may be such that the electrodes are spaced substantially away from the wall of the cardiac chamber. Intracardiac potential fields may be detected during a single cardiac beat. According to an example, the sensor electrodes may be distributed on a series of circumferences lying in planes spaced from each other. These planes may be perpendicular to the major axis of the end portion of the catheter. At least two additional electrodes may be provided adjacent at the ends of the major axis of the end portion. As a more specific example, the catheter may include four circumferences with eight electrodes spaced equiangularly on each circumference. Accordingly, in this specific implementation, the catheter may include at least 34 electrodes (32 circumferential and 2 end electrodes).

As example of electrical or cardiac mapping, an electrophysiological cardiac mapping system and technique based on a non-contact and non-expanded multi-electrode catheter (e.g., the catheter 110) can be implemented. ECGs may be obtained with one or more catheters 110 having multiple electrodes (e.g., such as between 42 to 122 electrodes). According to this implementation, knowledge of the relative geometry of the probe and the endocardium can be obtained by an independent imaging modality, such as transesophageal echocardiography. After the independent imaging, non-contact electrodes may be used to measure cardiac surface potentials and construct maps therefrom (e.g., in some cases using bipolar intracardiac reference signals). This technique can include the following steps (after the independent imaging step): (a) measuring electrical potentials with a plurality of electrodes disposed on a probe positioned in the heart 120; (b) determining the geometric relationship of the probe surface and the endocardial surface and/or other reference; (c) generating a matrix of coefficients representing the geometric relationship of the probe surface and the endocardial surface; and (d) determining endocardial potentials based on the electrode potentials and the matrix of coefficients.

As another example of electrical or cardiac mapping, a technique and apparatus for mapping the electrical potential distribution of a heart chamber can be implemented. An intra-cardiac multi-electrode mapping catheter assembly can be inserted into the heart 120. The mapping catheter (e.g., the catheter 110) assembly can include a multi-electrode array with one or more integral reference electrodes (e.g., one or the electrodes 111) or a companion reference catheter.

According to one or more embodiments, the electrodes may be deployed in the form of a substantially spherical array, which may be spatially referenced to a point on the endocardial surface by the reference electrode or by the reference catheter this is brought into contact with the endocardial surface. The preferred electrode array catheter may carry a number of individual electrode sites (e.g., at least 24). Additionally, this example technique may be implemented with knowledge of the location of each of the electrode sites on the array, as well as knowledge of the cardiac geometry. These locations are preferably determined by a technique of impedance plethysmography.

In view of electrical or cardiac mapping and according to another example, the catheter 110 can be a heart mapping catheter assembly that may include an electrode array defining a number of electrode sites. The heart mapping catheter assembly can also include a lumen to accept a reference catheter having a distal tip electrode assembly that may be used to probe the heart wall. The map heart mapping catheter assembly can include a braid of insulated wires (e.g., having x to y, such as 24 to 64, wires in the braid), and each of the wires may be used to form electrode sites. The heart mapping catheter assembly may be readily positioned in the heart 120 to be used to acquire electrical activity information from a first set of non-contact electrode sites and/or a second set of in-contact electrode sites.

Further, according to another example, the catheter 110 that can implement mapping electrophysiological activity within the heart can include a distal tip that is adapted for delivery of a stimulating pulse for pacing the heart or an ablative electrode for ablating tissue in contact with the tip. This catheter 110 can further include at least one pair of orthogonal electrodes to generate a difference signal indicative of the local cardiac electrical activity adjacent the orthogonal electrodes.

As noted herein, the system 100 can be utilized to detect, diagnose, and/or treat cardiac conditions. In example operation, a process for measuring electrophysiologic data in a heart chamber may be implemented by the system 100. The process may include, in part, positioning a set of active and passive electrodes into the heart 120, supplying current to the active electrodes, thereby generating an electric field in the heart chamber, and measuring the electric field at the passive electrode sites. The passive electrodes are contained in an array positioned on an inflatable balloon of a balloon catheter. In preferred embodiments, the array is said to have from x to y, such as 60 to 64, electrodes.

As another example operation, cardiac mapping may be implemented by the system 100 using one or more ultrasound transducers. The ultrasound transducers may be inserted into a patient's heart 120 and may collect a plurality of ultrasound slices (e.g., two dimensional or three-dimensional slices) at various locations and orientations within the heart 120. The location and orientation of a given ultrasound transducer may be known and the collected ultrasound slices may be stored such that they can be displayed at a later time. One or more ultrasound slices corresponding to the position of the probe 105 (e.g., a treatment catheter shown as catheter 110) at the later time may be displayed and the probe 105 may be overlaid onto the one or more ultrasound slices.

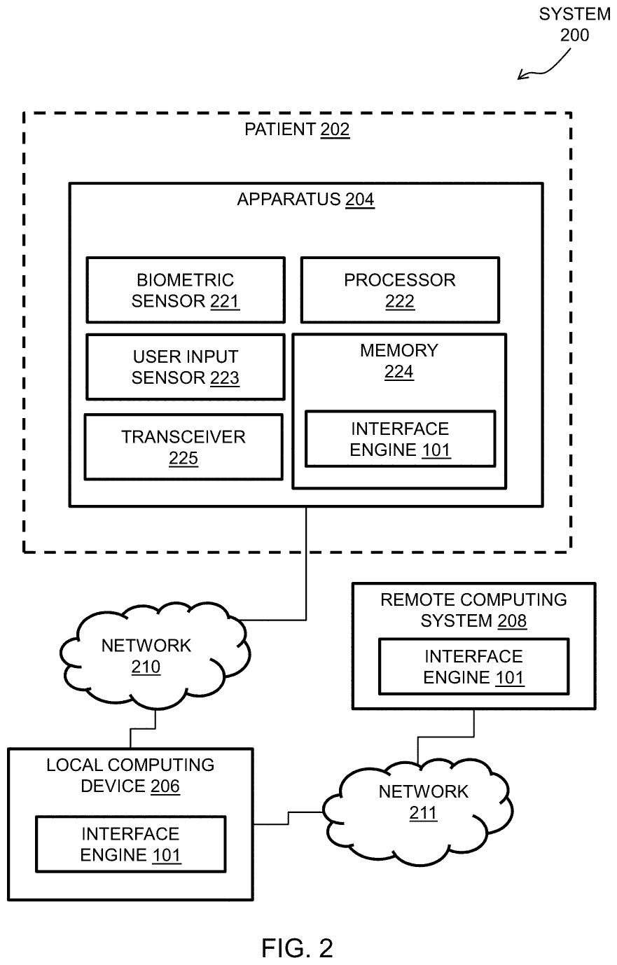

Turning now to FIG. 2, a diagram of a system 200 in which one or more features of the disclosure subject matter can be implemented is illustrated according to one or more embodiments. For instance, the system 200 is an example environment (e.g., medical device equipment, such as ENT navigation systems or other surgical systems) for implementing adaptive navigation and registration interface for medical imaging. The system 200 includes, in relation to a patient 202 (e.g., an example of the patient 125 of FIG. 1), an apparatus 204, a local computing device 206, a remote computing system 208, a first network 210, and a second network 211. Further, the apparatus 204 can include a biometric sensor 221 (e.g., an example of the catheter 110 of FIG. 1 or a surgical tool for ENT navigation systems), a processor 222, a user input (UI) sensor 223, a memory 224, and a transceiver 225. Note that the interface engine 101 of FIG. 1 is reused in FIG. 2 for ease of explanation and brevity. Additionally, the interface engine 101 of FIG. 2 can operate with respect to mapping any anatomical structure, body part, organ, or portion thereof.

According to an embodiment, the apparatus 204 can be an example of the system 100 of FIG. 1, where the apparatus 204 can include both components that are internal to the patient and components that are external to the patient. According to an embodiment, the apparatus 204 can be an apparatus that is external to the patient 202 that includes an attachable patch (e.g., that attaches to a patient's skin). According to another embodiment, the apparatus 204 can be internal to a body of the patient 202 (e.g., subcutaneously implantable), where the apparatus 204 can be inserted into the patient 202 via any applicable manner including orally injecting, surgical insertion via a vein or artery, an endoscopic procedure, or a lap aroscopic procedure. According to an embodiment, while a single apparatus 204 is shown in FIG. 2, example systems may include a plurality of apparatuses.

Accordingly, the apparatus 204, the local computing device 206, and/or the remote computing system 208 can be programed to execute computer instructions with respect the interface engine 101. As an example, the memory 224 stores these instructions for execution by the processor 222 so that the apparatus 204 can receive and process the biometric data via the biometric sensor 201. IN this way, the processor 22 and the memory 224 are representative of processors and memories of the local computing device 206 and/or the remote computing system 208.

The apparatus 204, local computing device 206, and/or the remote computing system 208 can be any combination of software and/or hardware that individually or collectively store, execute, and implement the interface engine 101 and functions thereof. Further, the apparatus 204, local computing device 206, and/or the remote computing system 208 can be an electronic, computer framework comprising and/or employing any number and combination of computing device and networks utilizing various communication technologies, as described herein. The apparatus 204, local computing device 206, and/or the remote computing system 208 can be easily scalable, extensible, and modular, with the ability to change to different services or reconfigure some features independently of others.

The networks 210 and 211 can be a wired network, a wireless network, or include one or more wired and wireless networks. According to an embodiment, the network 210 is an example of a short-range network (e.g., local area network (LAN), or personal area network (PAN)). Information can be sent, via the network 210, between the apparatus 204 and the local computing device 206 using any one of various short-range wireless communication protocols, such as Bluetooth, Wi-Fi, Zigbee, Z-Wave, near field communications (NFC), ultra-band, Zigbee, or infrared (IR). Further, the network 211 is an example of one or more of an Intranet, a local area network (LAN), a wide area network (WAN), a metropolitan area network (MAN), a direct connection or series of connections, a cellular telephone network, or any other network or medium capable of facilitating communication between the local computing device 206 and the remote computing system 208. Information can be sent, via the network 211, using any one of various long-range wireless communication protocols (e.g., TCP/IP, HTTP, 3G, 4G/LTE, or 5G/New Radio). Note that for either network 210 and 211 wired connections can be implemented using Ethernet, Universal Serial Bus (USB), RJ-11 or any other wired connection and wireless connections can be implemented using Wi-Fi, WiMAX, and Bluetooth, infrared, cellular networks, satellite or any other wireless connection methodology.

In operation, the apparatus 204 can continually or periodically obtain, monitor, store, process, and communicate via network 210 the biometric data associated with the patient 202. Further, the apparatus 204, local computing device 206, and/the remote computing system 208 are in communication through the networks 210 and 211 (e.g., the local computing device 206 can be configured as a gateway between the apparatus 204 and the remote computing system 208). For instance, the apparatus 204 can be an example of the system 100 of FIG. 1 configured to communicate with the local computing device 206 via the network 210. The local computing device 206 can be, for example, a stationary/standalone device, a base station, a desktop/laptop computer, a smart phone, a smartwatch, a tablet, or other device configured to communicate with other devices via networks 211 and 210. The remote computing system 208, implemented as a physical server on or connected to the network 211 or as a virtual server in a public cloud computing provider (e.g., Amazon Web Services (AWS)®) of the network 211, can be configured to communicate with the local computing device 206 via the network 211. Thus, the biometric data associated with the patient 202 can be communicated throughout the system 200.

Elements of the apparatus 204 are now described. The biometric sensor 221 may include, for example, one or more transducers configured to convert one or more environmental conditions into an electrical signal, such that different types of biometric data are observed/obtained/acquired. For example, the biometric sensor 221 can include one or more of an electrode (e.g., the electrode 111 of FIG. 1), a temperature sensor (e.g., thermocouple), a blood pressure sensor, a blood glucose sensor, a blood oxygen sensor, a pH sensor, an accelerometer, and a microphone.

The processor 222, in executing the interface engine 101, can be configured to receive, process, and manage the biometric data acquired by the biometric sensor 221, and communicate the biometric data to the memory 224 for storage and/or across the network 210 via the transceiver 225. Biometric data from one or more other apparatuses 204 can also be received by the processor 222 through the transceiver 225. Also, as described in more detail herein, the processor 222 may be configured to respond selectively to different tapping patterns (e.g., a single tap or a double tap) received from the UI sensor 223, such that different tasks of a patch (e.g., acquisition, storing, or transmission of data) can be activated based on the detected pattern. In some embodiments, the processor 222 can generate audible feedback with respect to detecting a gesture.

The UI sensor 223 includes, for example, a piezoelectric sensor or a capacitive sensor configured to receive a user input, such as a tapping or touching. For example, the UI sensor 223 can be controlled to implement a capacitive coupling, in response to tapping or touching a surface of the apparatus 204 by the patient 202. Gesture recognition may be implemented via any one of various capacitive types, such as resistive capacitive, surface capacitive, projected capacitive, surface acoustic wave, piezoelectric and infra-red touching. Capacitive sensors may be disposed at a small area or over a length of the surface, such that the tapping or touching of the surface activates the monitoring device.

The memory 224 is any non-transitory tangible media, such as magnetic, optical, or electronic memory (e.g., any suitable volatile and/or non-volatile memory, such as random-access memory or a hard disk drive). The memory 224 stores the computer instructions for execution by the processor 222.

The transceiver 225 may include a separate transmitter and a separate receiver. Alternatively, the transceiver 225 may include a transmitter and receiver integrated into a single device.

In operation, the apparatus 204, utilizing the interface engine 101, observes/obtains the biometric data of the patient 202 via the biometric sensor 221, stores the biometric data in the memory, and shares this biometric data across the system 200 via the transceiver 225. The interface engine 101 can then utilize models, neural networks, machine learning, and/or artificial intelligence to aggregate data from completed cases, analyze the data, and output grades for the data, and therefore provide recommendations based on the graded data.

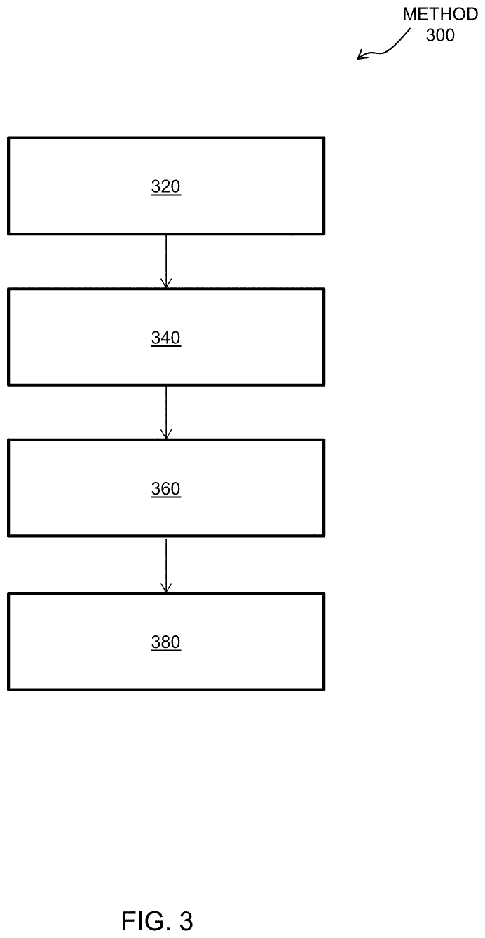

Turning now to FIG. 3, a method 300 (e.g., performed by the interface engine 101 of FIG. 1 and/or of FIG. 2) is illustrated according to one or more exemplary embodiments. For ease of explanation, the method 300 as implemented by the interface engine 101 is described herein with respect to ENT navigation and registration; however, any anatomical structure, body part, organ, or portion thereof can be a target for mapping by the interface engine 101. The method 300 addresses limits of present GUIs by providing a multi-step manipulation of cases and data that enables an improved understanding an electrophysiology with more precision through an adaptive navigation and registration interface for medical imaging. More particularly, the method 300 is an example of establishing a database of graded procedures to improve understanding of ENT navigation and registration.

The method 300 begins at block 320, where the interface engine 101 aggregates data from one or more completed cases. The completed cases can include, but are not limited to, CT scans and/or MR scans respective to medical treatments, surgical plans, surgical procedures, or medicals diagnoses performed by operations of the interface engine 101. For example, the completed cases can include all ENT navigation and registration procedures relative to CT and MR scans. Navigation can include a process of determining a location (e.g., an x-y-z coordinate) with respect to an anatomical structure. Registration can include a process of acquiring and maintaining information at each location. The data of each completed case can include, but is not limited to, the location and registration information, along with case type, average environmental interference, errors, surgical measurements, biometric data, user data, historical data, and diagnosis data associated with the completed case (e.g., an outcome of the ENT navigation and registration procedure).

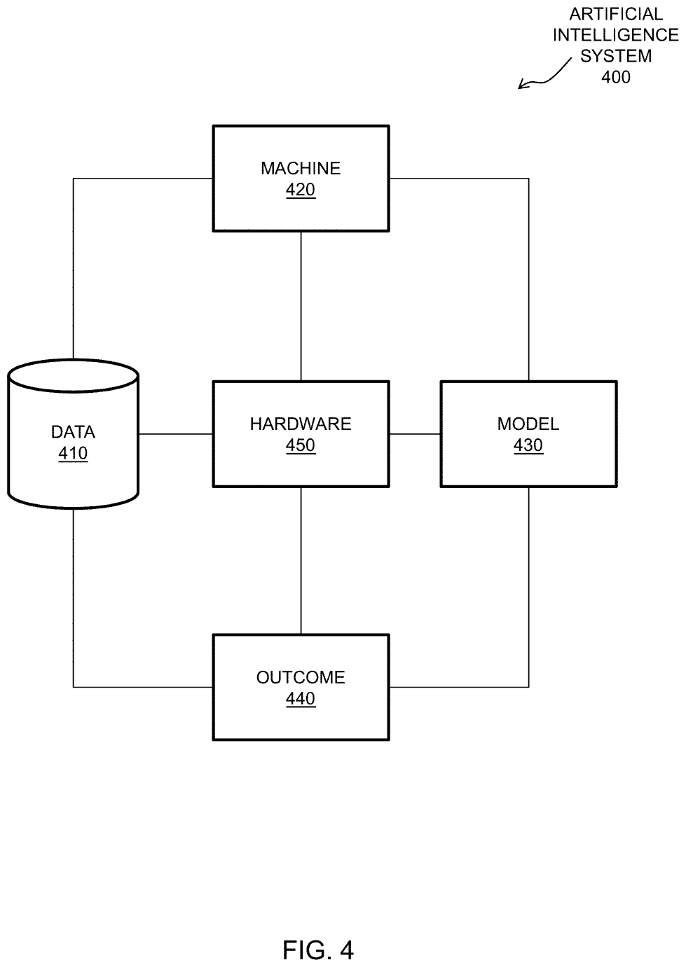

Turning to FIG. 4, a graphical depiction of an artificial intelligence system 400 implemented by the interface engine 101 is illustrated according to one or more embodiments. The artificial intelligence system 400 includes data 410 (e.g., data from one or more completed cases), a machine 420, a model 430, an outcome 440, and (underlying) hardware 450. Note that the machine 410, the model 430, and the hardware 450 can represent aspects of the interface engine 101 of FIGS. 1-2 (e.g., machine learning and/or an artificial intelligence algorithm therein), while the hardware 450 can also represent the catheter 110 of FIG. 1, the console 160 of FIG. 1, and/o the apparatus 204 of FIG. 2.

In general, the machine learning and/or the artificial intelligence algorithms of the artificial intelligence system 400 (e.g., as implemented by the interface engine 101 of FIGS. 1-2 and the method 300 of FIG. 3) operate with respect to the hardware 450, using the data 410, to train the machine 420, build the model 430, and predict the outcomes 440.

For instance, with respect to FIG. 4, the machine 420 operates as a controller to provide data collection associated with the hardware 450 (e.g., aggregates data at block 320 of FIG. 3). The data 410 (e.g., data from one or more completed cases of block 320 of FIG. 3) can be on-going, stored, and/or outputted location and registration information associated with the hardware 450. According to one or more embodiments, the data 410 can include location and registration information acquired during an ENT navigation and registration procedure. The data 410 can be divided by the machine 420 into one or more subsets.

Returning to FIG. 3, at block 340, the interface engine 101 analyzes the data. According to one or more embodiments, the interface engine 101 can then utilize models, neural networks, machine learning, and/or artificial intelligence to analyze the data. The analysis determines one or more of accuracy, consistency, and error within and/or across the one or more completed cases.

At block 360, the interface engine 101 outputs/generates grades (e.g., the outcomes 440) for the data (e.g., the data 410). In this way, the data can be analyzed to produce one or more grades. A single grade can rank and/or score accuracy, consistency, and error of an instance of the location information or the registration information. A single grade can also rank and/or score accuracy, consistency, and error the entirety of the completed case. Examples of one or more grades include an alphanumeric character selected from a range identifying accuracy, a percentage of points (e.g., locations) that during a completed case were in a “no-fly” zone (e.g., identifying whether bone was consistently crossed during registration or with a tool), and/or a color coding identifying a degree of error. Note that the grades can be outputted to a display. Note that the interface engine 101 can classify segmentations as allowed zones (e.g., air, tissue) and “no-fly zones” (e.g., bone tissue that is never removed during similar cases, where the assumption is never being able to go through some of the bone). When the interface engine 101 identifies that a navigated location is inside the no-fly zone, then there is an inaccuracy (e.g., which may cause a case to be analyzed overall, and all navigated locations to be checked to see which parts experienced inaccuracies).

As an example, in view of FIG. 4, the machine 420 trains, such as with respect to the hardware 450. This training can also include an analysis and correlation of the data 410 collected to grade the data 410. Each grade (e.g., the outcomes 440) can include, but are not limited to, ‘how well’ navigation and registration went for each completed case. For example, in the case of the ENT navigation and registration procedure, the data 410 with respect to corresponding outcomes can be trained to determine if a correlation or link exists between different ENT navigation and registration procedures. Moreover, the model 430 is built on the data 410 associated with the hardware 450. Building the model 430 can include physical hardware or software modeling, algorithmic modeling, and/or the like that seeks to represent the data 410 (or subsets thereof) that has been collected and trained. In some aspects, building of the model 430 is part of self-training operations by the machine 420.

Further, the model 430 can be configured to model the operation of hardware 450 and model the data 410 collected from the hardware 450 to predict the outcome 440 achieved by the hardware 450. Predicting the outcomes 440 (of the model 430 associated with the hardware 450) can utilize a trained model 430. Thus, using the outcome 440 that is predicted, the machine 420, the model 430, and the hardware 450 can be configured accordingly.

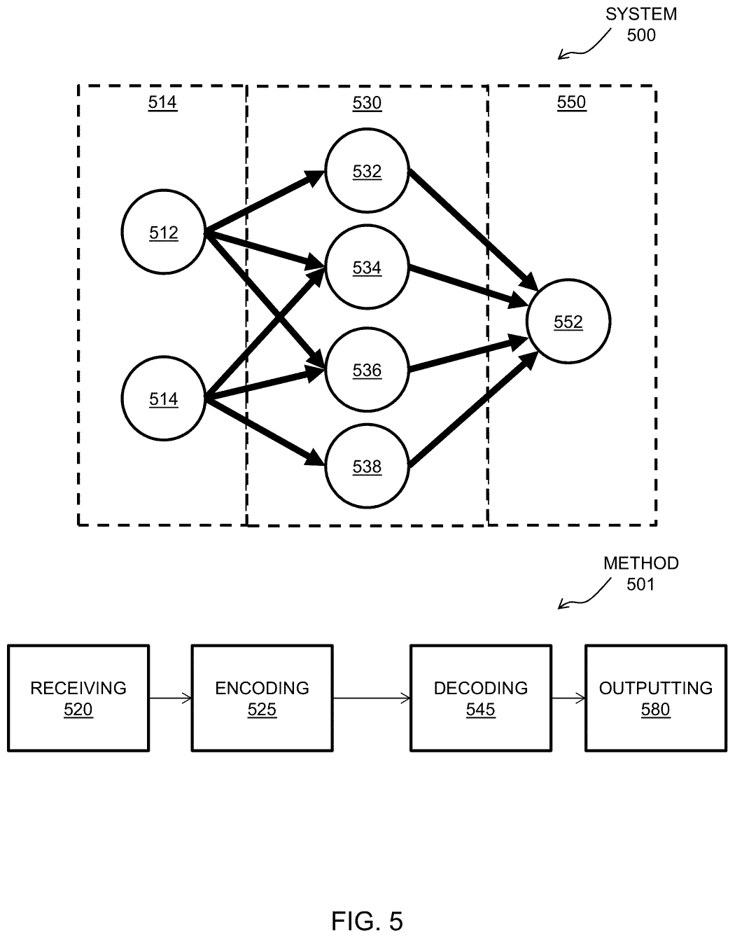

At block 380, the interface engine 101 stores the grades to establish a database of graded cases (e.g., 1000+ cases from a medical center) and for presentation in a GUI of the interface engine 101. The grades are stored in conjunction with the complete case and data thereof, whether in a local memory or elsewhere. Note that storage enables further analysis, while the GUI of the interface engine 101 and the grades provide enhanced interface features, as described with respect to FIGS. 5-11.

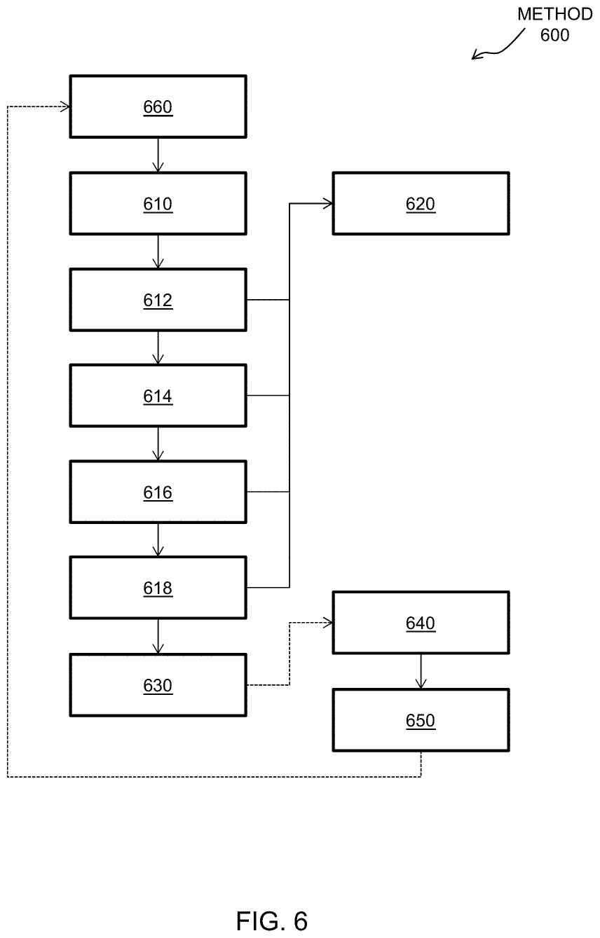

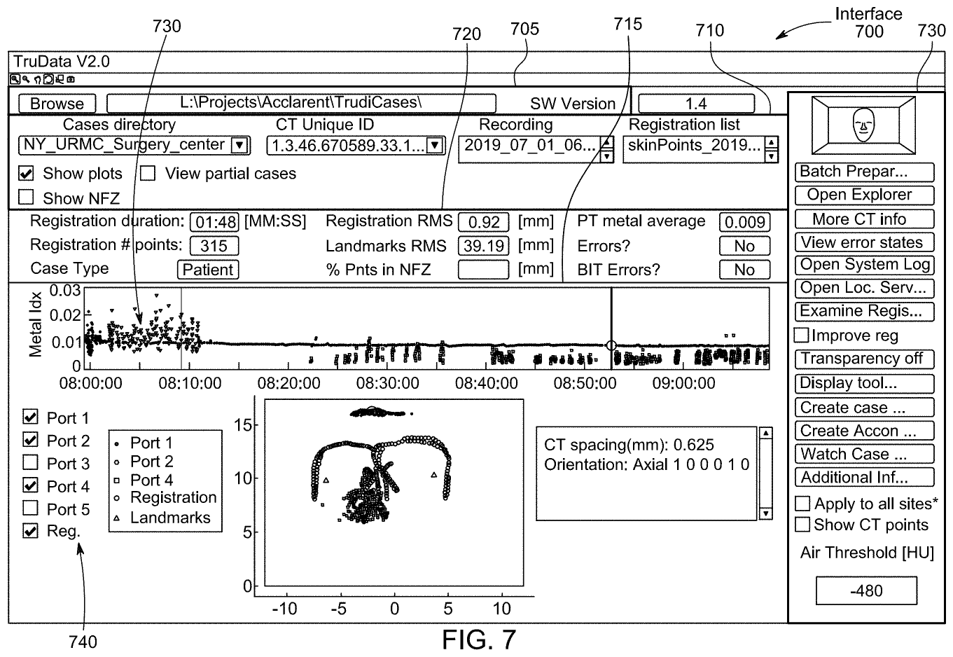

Turning now to FIGS. 5-11, an example ENT navigation and registration procedure implemented by the interface engine 101 is described according to one or more embodiments. FIG. 5 illustrates an example of a neural network 500 and a block diagram of a method 501 performed in the neural network 500 according to one or more embodiments. FIG. 6 illustrates a method 300, performed by the interface engine 101 using the neural network 500, according to one or more exemplary embodiments. In this way, the neural network 500 operates to support implementation of the machine learning and/or the artificial intelligence algorithms of the interface engine 101. FIGS. 7-11 illustrate example interfaces generated by the interface engine 101 according to one or more embodiments.

In general, the interface engine 101 by implementing the method 600 provides a GUI with enhanced data and visualization, such as grades with respect to navigation and registration. In this way, the physician 115 can utilize the GUI to confirm in real time whether a particular point, such as a frontal sinus, was reached during a navigation procedure. Further, if the physician 115 is not certain, the interface engine 101 can quickly access data to double-check, based on a user-friendly arrangement of features, options and functionality displayed by the GUI. Additionally, once the interface engine 101 establishes a database, the interface engine 101 can provide procedural recommendation to the physician 115.

The method 600 begins at block 610, where the interface engine 101 initiates a case. The cases can include an ENT navigation and registration procedure. Initiating a case can include performing CT scans and/or MR scans in support of the ENT navigation and registration procedure. In this way, a map produced from the CT scans and/or MR scans can be used during the ENT navigation and registration procedure.

At block 612, the interface engine 101 receives navigation information from a tool in real-time to determine locations (e.g., x-y-z coordinates) with respect to an anatomical structure being examined (e.g., an interior of a nose). At block 614, the interface engine 101 receives registration information from the tool in real-time. At block 616, additional information, such as surgical measurements, biometric data, user data, historical data, and diagnosis data, can be associated with the case.

At block 618, the interface engine 101 analyzes the navigation, registration, and additional information to generate one or more grades. A grade can be an evaluation both the registration and navigation accuracy on a CT or MRI, along with indicate an accuracy of the tool used, a consistency of the measurements, and an error in the procedure (e.g., a likelihood of interference).

According to one or more embodiments, the interface engine 101 can utilize big data (e.g., 1000+ cases from a medical center) to evaluate parameters that are important to a specific site (e.g., the medical center). For instance, how much metal interference exists during registration or during the case itself and does the big data indicate consistent problems (e.g., does the medical center show consistent metal interference in the information) are questions the one or more grades can identify. That it, metal interference affects accuracy and/or results. A low grade may indicate that a particular medical center may have a metal tray of tools too close to a patient 125. In turn, the interface engine 101 can analyzes the present case (at block 618) in comparison with the big data to generate a grade with respect to metal interference.

In an example operation, the interface engine 101 of FIG. 1 includes collecting the information into the neural network 500. An input layer 510 is represented by a plurality of inputs (e.g., inputs 512 and 514 of FIG. 5). With respect to block 520 of the method 501, the input layer 510 receives the inputs 512 and 514. The inputs 512 and 514 can include the navigation, registration, and additional information of blocks 612, 614, and 616.

At block 525 of the method 501, the neural network 500 encodes the inputs 512 and 514 utilizing any portion of the navigation, registration, and additional information to produce a latent representation or data coding. The latent representation includes one or more intermediary data representations derived from the plurality of inputs. According to one or more embodiments, the latent representation is generated by an element-wise activation function (e.g., a sigmoid function or a rectified linear unit) of the interface engine 101 of FIG. 1. As shown in FIG. 5, the inputs 512 and 514 are provided to a hidden layer 530 depicted as including nodes 532, 534, 536, and 538. The neural network 500 performs the processing via the hidden layer 530 of the nodes 532, 534, 536, and 538 to exhibit complex global behavior, determined by the connections between the processing elements and element parameters. Thus, the transition between layers 510 and 530 can be considered an encoder stage that takes the inputs 512 and 514 and transfers it to a deep neural network (within layer 530) to learn some smaller representation of the input (e.g., a resulting the latent representation).

The deep neural network can be a convolutional neural network (CNN), a long short-term memory neural network, a fully connected neural network, or combination thereof. This encoding provides a dimensionality reduction of the inputs 512 and 514. Dimensionality reduction is a process of reducing the number of random variables (of the inputs 512 and 514) under consideration by obtaining a set of principal variables. For instance, dimensionality reduction can be a feature extraction that transforms data (e.g., the inputs 512 and 514) from a high-dimensional space (e.g., more than 10 dimensions) to a lower-dimensional space (e.g., 2-3 dimensions). Accordingly, one or more advantages, technical effects, and benefits of dimensionality reduction include reducing time and storage space requirements for the data, improving visualization of the data, and improving parameter interpretation for machine learning. This data transformation can be linear or nonlinear. The operations of receiving (block 520) and encoding (block 525) can be considered a data preparation portion of the multi-step data manipulation by the interface engine 101.

At block 545 of the method 510, the neural network 500 decodes the latent representation. The decoding stage takes the encoder output (e.g., the resulting the latent representation) and attempts to reconstruct some form of the inputs 512 and 514 using another deep neural network. In this regard, the nodes 532, 534, 536, and 538 are combined to produce in the output layer 550 an output 552, as shown in block 560 of the method 510. That is, the output layer 590 reconstructs the inputs 512 and 514 on a reduced dimension but without the signal interferences, signal artifacts, and signal noise. Examples of the output 552 include cleaned navigation, registration, and additional information (e.g., clean/denoised version thereof), along with the one or more grades.

Returning to FIG. 6, at block 620, the interface engine 101 presents the information and the one or more grades during the ENT navigation and registration procedure. Each grade (e.g., the outcomes 440) can include, but are not limited to, ‘how well’ navigation and registration is going. That is, as the data is being graded, the interface engine 101 presents the one or more grades. In this regard, during a present case, the physician 115 can receive immediate feedback and/or warning. For instance, the grade can be a percentage of points that during a case were in a “no-fly” zone (e.g., bone is crossed during registration or with a tool).

With respect to the interface engine 101 presenting the information and the one or more grades during the ENT navigation and registration procedure, FIG. 7 illustrates an exemplary interface 700 according to one or more embodiments. The includes at least frames 705, 710, 715, 720, and 730. The frame 705 provides a data folder display, while the frame 710 provides explorer and user interface options. The explorer option presents valid cases and registrations for a selected site. The user interface options are also provided so that the physician 125 may choose whether to show/hide plots, partial cases in the explorer option, and/or to superimpose the data classified as “no-fly zone” from the patient's scan.

The frame 715 provides plots. For instance, a first plot may show a timeline of the case, where each color represents a different tool (e.g., port) that was used. The frame 715 also enables the physician 125 to see a second plot, such as the magnetic interference of each tool in relation to where the tool was shown at a particular point in time. Further, a line and marker 740 show dynamic viewing of each of the tools, which the checkboxes 750 show which port is active (e.g., enabled/disabled).

The frame 720 provides general details about a given case, such as registration information, case type, percentage of points, average environmental interference, and errors (e.g., the one or more grades). Registration information can include duration, a number of points acquired, root means square (“RMS”), and RMS of landmarks after registration. Note that the RMS and the RMS of landmarks after registration are metrics of registration accuracy. Case type can indicate whether the given case is a patient case, a head model, or a simulated use test. The percentage of points that crossed a “no-fly zone” indicates navigation accuracy (this feature can be enabled and disabled). The average environmental interference can be on a patient tracker. The indication of whether system errors were experienced during the case can include whether errors are related to communication between the tools and the system.

The frame 730 provides advanced options in a list of sub-menus, such as replay case and create reports, a troubleshoot tool, and examine registration.

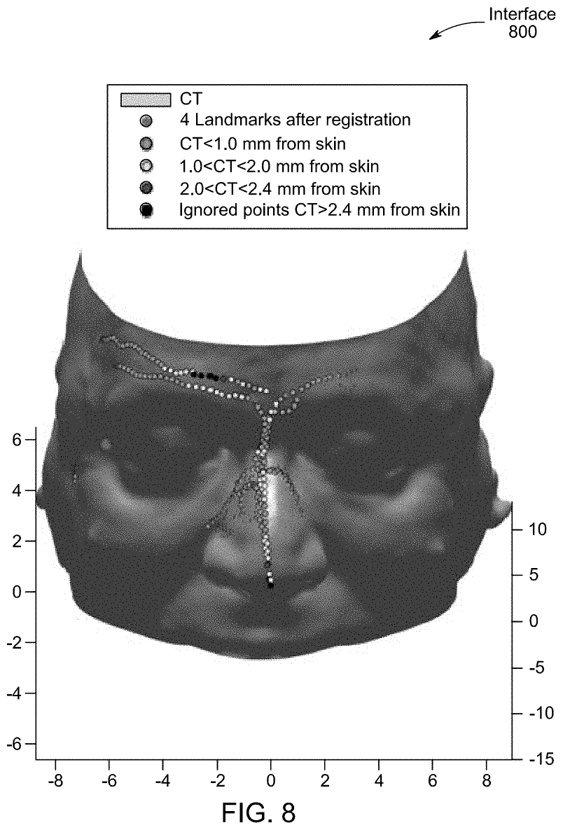

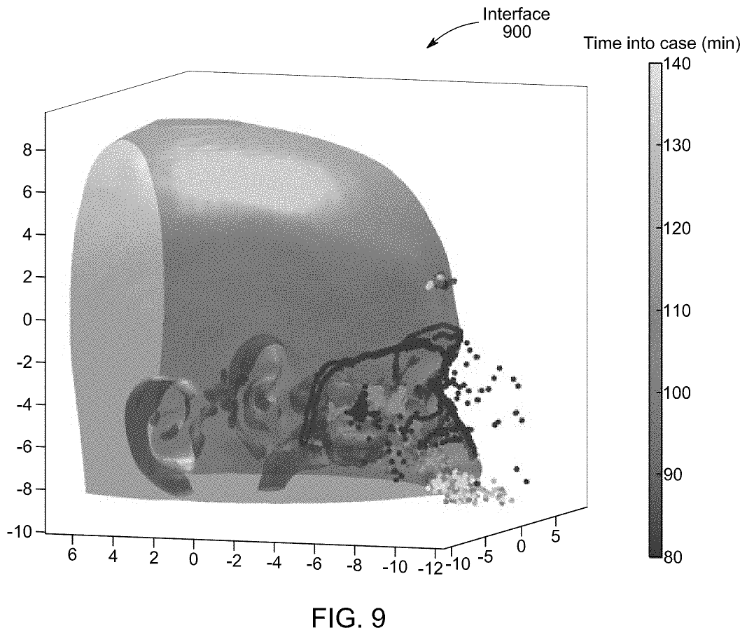

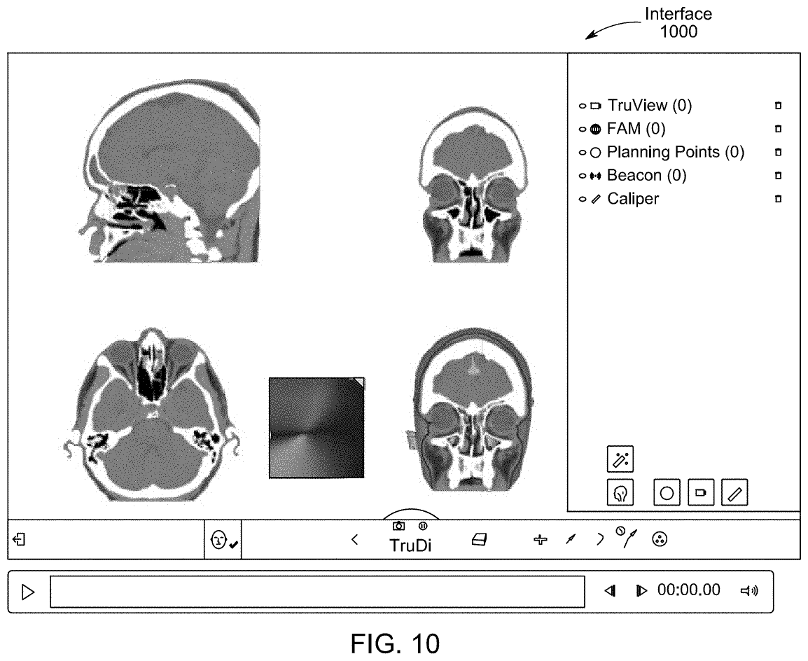

With further respect to the interface engine 101 presenting the information and the one or more grades during the ENT navigation and registration procedure, FIGS. 8-10 illustrates exemplary interfaces according to one or more embodiments. FIG. 8 shows a graphical presentation 800 of the ENT registration procedure, including color coding of registration quality. The graphical presentation 800 is a registration superimposed on a three dimensional CT scan, so that the registration quality can be presented and evaluated. FIG. 9 shows a navigation map 900, which can be color coded by time or size coded by type of tool (e.g., to show everywhere the tools have been inside the anatomy). FIG. 10 shows a screen shot 1000 of a movie, which summarizes an entire case (e.g., in 7 seconds) and shows a 360 degree panorama of the navigation map 900. Note that the interface engine 101 provides replay of a chosen case.

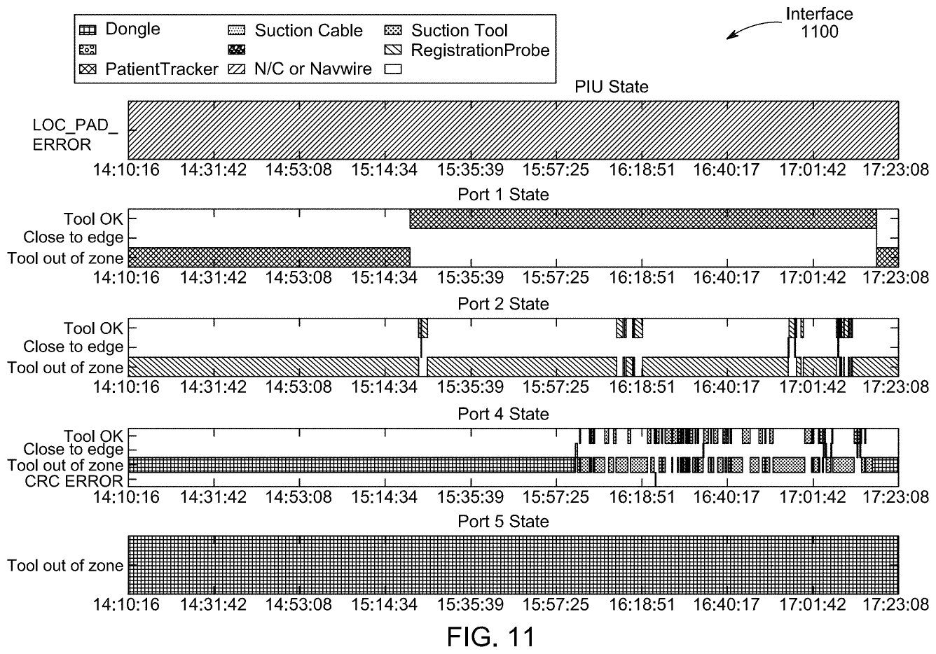

At block 625, the interface engine 101 receives user feedback. In this regard, the physician 115 can interact with the GUI of the interface engine 101 to evaluate a given case. For instance, errors can be presented to the physician 115. FIG. 11 illustrates an exemplary interface 1100 according to one or more embodiments, where errors during a case are tracked on a timeline that includes separate plots to detail the actions of the various tools used during the case. According to one or more embodiments, the interface engine 101 can accommodate preferences of the physician 115. For example, the physician 115 may optionally select which features to include on the GUI or in which location or arrangement a particular feature will be positioned on the GUI.

At block 630, once the case is completed, the interface engine 101 provides the case and all associated data and grades for storage. The grades are stored in conjunction with the complete case and data thereof, whether in a local memory or elsewhere. Storage enabled further analysis. According to one or more embodiments, an advanced feature of the interface engine 101 includes creating a report either per site (e.g., medical center, hospital, or clinic) or a database. Using this report, the interface engine 101 allows analysis and comparison of data from different surgeons, different hospitals, or different geographical regions. The data included in the report contains, but is not limited to case duration, CT properties, tools used, features used, information about registration, system errors that were experienced, and ferromagnetic interference.

At block 640, big data analysis. In this regard, the interface engine 101 curates and analyzes the database of cases based on one or more of different surgeons, different hospitals, or different geographical regions for best practices (e.g., determine what has worked in the past before beginning a new case).

At block 650, generate procedural recommendations, which can be presented at block 660 before block 610. Additionally, based on the graded data in the database, the interface engine 101 can suggest user-specific guidance (e.g., how to improve registration if registration grade is low, or what to do if accuracy has degraded during case).

According to one or more embodiments, a neural network is a network or circuit of neurons, or in a modern sense, an artificial neural network (ANN), composed of artificial neurons or nodes or cells.

For example, an ANN involves a network of processing elements (artificial neurons) which can exhibit complex global behavior, determined by the connections between the processing elements and element parameters. These connections of the network or circuit of neurons are modeled as weights. A positive weight reflects an excitatory connection, while negative values mean inhibitory connections. Inputs are modified by a weight and summed using a linear combination. An activation function may control the amplitude of the output. For example, an acceptable range of output is usually between 0 and 1, or it could be −1 and 1. In most cases, the ANN is an adaptive system that changes its structure based on external or internal information that flows through the network.

In more practical terms, neural networks are non-linear statistical data modeling or decision-making tools that can be used to model complex relationships between inputs and outputs or to find patterns in data. Thus, ANNs may be used for predictive modeling and adaptive control applications, while being trained via a dataset. Note that self-learning resulting from experience can occur within ANNs, which can derive conclusions from a complex and seemingly unrelated set of information. The utility of artificial neural network models lies in the fact that they can be used to infer a function from observations and also to use it. Unsupervised neural networks can also be used to learn representations of the input that capture the salient characteristics of the input distribution, and more recently, deep learning algorithms, which can implicitly learn the distribution function of the observed data. Learning in neural networks is particularly useful in applications where the complexity of the data (e.g., the biometric data) or task (e.g., monitoring, diagnosing, and treating any number of various diseases) makes the design of such functions by hand impractical.

Neural networks can be used in different fields. Thus, the machine learning and/or the artificial intelligence algorithms therein can include neural networks that are divided generally according to tasks to which they are applied. These divisions tend to fall within the following categories: regression analysis (e.g., function approximation) including time series prediction and modeling; classification including pattern and sequence recognition; novelty detection and sequential decision making; data processing including filtering; clustering; blind signal separation, and compression. For example, Application areas of ANNs include nonlinear system identification and control (vehicle control, process control), game-playing and decision making (backgammon, chess, racing), pattern recognition (radar systems, face identification, object recognition), sequence recognition (gesture, speech, handwritten text recognition), medical diagnosis and treatment, financial applications, data mining (or knowledge discovery in databases, “KDD”), visualization and e-mail spam filtering. For example, it is possible to create a semantic profile of patient biometric data emerging from medical procedures.

According to one or more embodiments, the neural network can implement a long short-term memory neural network architecture, a CNN architecture, or other the like. The neural network can be configurable with respect to a number of layers, a number of connections (e.g., encoder/decoder connections), a regularization technique (e.g., dropout); and an optimization feature.

The long short-term memory neural network architecture includes feedback connections and can process single data points (e.g., such as images), along with entire sequences of data (e.g., such as speech or video). A unit of the long short-term memory neural network architecture can be composed of a cell, an input gate, an output gate, and a forget gate, where the cell remembers values over arbitrary time intervals and the gates regulate a flow of information into and out of the cell.

The CNN architecture is a shared-weight architecture with translation invariance characteristics where each neuron in one layer is connected to all neurons in the next layer. The regularization technique of the CNN architecture can take advantage of the hierarchical pattern in data and assemble more complex patterns using smaller and simpler patterns. If the neural network implements the CNN architecture, other configurable aspects of the architecture can include a number of filters at each stage, kernel size, a number of kernels per layer.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Although features and elements are described above in particular combinations, one of ordinary skill in the art will appreciate that each feature or element can be used alone or in any combination with the other features and elements. In addition, the methods described herein may be implemented in a computer program, software, or firmware incorporated in a computer-readable medium for execution by a computer or processor. A computer readable medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire

Examples of computer-readable media include electrical signals (transmitted over wired or wireless connections) and computer-readable storage media. Examples of computer-readable storage media include, but are not limited to, a register, cache memory, semiconductor memory devices, magnetic media such as internal hard disks and removable disks, magneto-optical media, optical media such as compact disks (CD) and digital versatile disks (DVDs), a random access memory (RAM), a read-only memory (ROM), an erasable programmable read-only memory (EPROM or Flash memory), a static random access memory (SRAM), and a memory stick. A processor in association with software may be used to implement a radio frequency transceiver for use in a terminal, base station, or any host computer.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one more other features, integers, steps, operations, element components, and/or groups thereof.

The descriptions of the various embodiments herein have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method implemented by an interface engine stored as processor executable code on a memory coupled to one or more processors, the processor executable code being executed by the one or more processors, the method comprising:
aggregating, by the interface engine, data from one or more completed cases, the data comprising location information and registration information, the one or more completed cases comprising at least one ear, nose, and throat navigation and registration procedure;
analyzing, by the interface engine using machine learning an artificial intelligence, the data for accuracy, consistency, or error within or across the one or more completed cases; and
generating, by the interface engine, one or more grades based on the analysis of the data.

2. The method of claim 1, wherein the one or more completed cases comprises one or more of medical treatments, surgical plans, surgical procedures, and medicals diagnoses.

3. The method of claim 1, wherein the navigation information comprises x-y-z coordinate information with respect to an anatomical structure.

4. The method of claim 3, wherein the anatomical structure comprises an interior of a nose.

5. The method of claim 1, wherein the registration information comprises one or more of surgical measurements, biometric data, user data, historical data, and diagnosis data associated with the at least one ear, nose, and throat navigation and registration procedure.

6. The method of claim 1, wherein the one or more grades rank or score an instance of the location information or the registration information.

7. The method of claim 1, wherein the one or more grades rank or score the at least one ear, nose, and throat navigation and registration procedure.

8. The method of claim 1, wherein the one or more grades identify how well navigation and registration went for each completed case with respect to corresponding outcomes and whether if a correlation exists between the one or more completed cases.

9. The method of claim 1, wherein the interface engine receives additional data from an initiation of associated with the at least one ear, nose, and throat navigation and registration procedure.

10. The method of claim 1, wherein the interface engine presents the one or more grades during a current procedure in view of the one or more completed cases.

11. A system comprising
a memory storing executable code an interface engine; and
one or more processors coupled to the memory, the one or more processors configured to execute the processor executable code to cause the system to perform:
aggregating, by the interface engine, data from one or more completed cases, the data comprising location information and registration information, the one or more completed cases comprising at least one ear, nose, and throat navigation and registration procedure;
analyzing, by the interface engine using machine learning an artificial intelligence, the data for accuracy, consistency, or error within or across the one or more completed cases; and
generating, by the interface engine, one or more grades based on the analysis of the data.

12. The system of claim 1, wherein the one or more completed cases comprises one or more of medical treatments, surgical plans, surgical procedures, and medicals diagnoses.

13. The system of claim 1, wherein the navigation information comprises x-y-z coordinate information with respect to an anatomical structure.

14. The system of claim 13, wherein the anatomical structure comprises an interior of a nose.

15. The system of claim 1, wherein the registration information comprises one or more of surgical measurements, biometric data, user data, historical data, and diagnosis data associated with the at least one ear, nose, and throat navigation and registration procedure.

16. The system of claim 1, wherein the one or more grades rank or score an instance of the location information or the registration information.

17. The system of claim 1, wherein the one or more grades rank or score the at least one ear, nose, and throat navigation and registration procedure.

18. The system of claim 1, wherein the one or more grades identify how well navigation and registration went for each completed case with respect to corresponding outcomes and whether if a correlation exists between the one or more completed cases.

19. The system of claim 1, wherein the interface engine receives additional data from an initiation of associated with the at least one ear, nose, and throat navigation and registration procedure.

20. The system of claim 1, wherein the interface engine presents the one or more grades during a current procedure in view of the one or more completed cases.
